(19) United States
(12) Patent Application Publication
(10) Pub. No.: US 2018/0116926 A1
(43) Pub. Date: May 3, 2018

(54) SUNLESS TANNING COMPOSITION COMPRISING SOLUBLE AND INSOLUBLE SILICONE-CONTAINING POLYMERS

(71) Applicant: Australian Gold, LLC, Indianapolis, IN (US)

(72) Inventors: Maxine Johnson, Arizona City, AZ (US); George Franklin Feldman, III, Indianapolis, IN (US); Angie H. Provo, Carmel, IN (US); Emily Qadri, Avon, IN (US)

(73) Assignee: Australian Gold, LLC, Indianapolis, IN (US)

(21) Appl. No.: 15/797,822

(22) Filed: Oct. 30, 2017

Related U.S. Application Data
(60) Provisional application No. 62/496,857, filed on Oct. 31, 2016.

(51) Int. Cl. A61K 8/35; A61Q 19/04; A61K 8/44; A61K 8/49; A61K 8/34; A61K 8/9728; A61K 8/9789; A61K 8/365; A61K 8/894; A61K 8/92; A61M 35/00; A61M 11/00

(52) U.S. Cl. CPC A61K 8/35; A61M 2210/04; A61K 8/44; A61K 8/4953; A61K 8/345; A61K 8/9728; A61K 8/9789; A61K 8/365; A61K 8/894; A61K 8/922; A61K 8/4973; A61M 35/00; A61M 11/006; A61K 2800/805; A61Q 19/04

(57) ABSTRACT

In certain aspects, the present disclosure relates to sunless tanning compositions and methods for using the same. It has been found that using a combination of water soluble silicones and/or water dispersible silicones may improve the deposition of certain cosmetic ingredients and/or form a protective layer over certain cosmetic ingredients. Such improved sunless tanning compositions may reduce perception of a tacky or sticky feeling after deposition of the sunless tanning composition.

### CROSS-REFERENCE TO RELATED APPLICATION

This application claims the benefit of U.S. Provisional Application No. 62/496,857 filed Oct. 31, 2016, which is hereby incorporated by reference in its entirety.

### FIELD OF THE DISCLOSURE

The present disclosure is in the field of sunless tanning compositions.

### BACKGROUND

Sunless tanning compositions are a popular cosmetic product. Sunless tanning compositions can, for example, impart color to the skin of a human without, or with reduced, exposure to sun or artificial UV light. Many people use sunless tanning compositions to feel good and to project the image of a healthy or active lifestyle. The resulting skin color may help them look and feel healthier and thinner.

Often, sunless tanning compositions are formulated from a variety of ingredients. Some sunless tanning compositions may suffer from uneven color distribution on skin as well as coloring that may not aesthetically enhance the best preferred color of naturally tanned skin for a variety of skin types and/or skin tones.

Sunless tanning compositions typically last for only a limited period of time, for example a few days. The time the color lasts on the skin may be reduced by many factors including, but not limited to, rubbing of the skin, washing of the skin, and/or unfavorable conditions of the skin such as dry skin.

Conventional wisdom in the tanning industry teaches that it is undesirable to include silicone in formulations of sprayable sunless tanning compositions. Correspondingly, conventional wisdom is that sprayable sunless tanning compositions should not be applied after other cosmetic compositions that may contain a silicone ingredient. For example, many indoor tanning compositions used with ultraviolet tanning beds contain silicone ingredients. When silicone containing compositions are used in or before a sprayable sunless tanning composition, the silicone may reduce or block the DHA or other active sunless agents from being absorbed by the skin and therefore reduces the ability to achieve a sunless tan.

Sunless tanning compositions may be applied by a variety of methods, however, two major markets have appeared in the sunless tanning space: (1) salon products and/or commercial-grade machines; and (2) products which may be sold directly to the consumer for use and/or application by the consumer.

Typically, consumer sunless tanning compositions are sold in smaller containers where the container is designed for the product to be manually applied by various methods. Forms for application include pressurized spray cans, manual pump sprays, lotions, creams, impregnated towelettes or gloves, and other forms typically used for applying personal cosmetics. One method of applying consumer sunless tanning compositions is using “bag on valve” or “BOV” apparatuses. Bag on valve apparatuses typically comprise an aerosol valve welded to a bag placed inside a can. The sunless tanning composition is placed inside the bag, while the propellant is placed in the space outside of the bag but inside of the can. A consumer can use the BOV apparatus to spray the sunless tanning composition onto their skin.

Salon and/or commercial application methods typically include spraying a sunless tanning composition on the skin of a human using an automated machine such as spray booth or a human operated machine such as an air brush apparatus. Commercial-grade sunless tanning booths may include one or more automated mechanized arms with one or more nozzles which can spray a sunless tanning composition on to the skin. The spray is typically generated using a powered air compressor. These commercial-grade tanning booths may be operated manually by an operator who controls the machine, and/or may be operated by a microcontroller, microcomputer, and/or computer to perform automated application on to the skin of a human.

Commercial-grade tanning booths may comprise one or more nozzles which can spray a composition on to the skin in the form of a fine mist, an aerosol, and/or larger droplets sizes if desired. In some cases the one or more nozzles may oscillate and/or move relative to the tanning booth and/or relative to person receiving the sunless tanning composition. Typically, these commercial-grade sunless tanning booths cost thousands of dollars. Some include the option of delivering one or more sunless tanning compositions that can be applied without recharging the machine. For example, such commercial-grade sunless tanning booths can employ containers which can be loaded in bulk, for example but not limited to containers that can hold about one gallon and/or larger volumes. Alternatively, commercial-grade sunless tanning booths may employ single use cans or cartridges that can be used in a sunless tanning booth at the salon. This may allow the customer to use their preferred sunless tanning composition.

In light of the background above, there remains a need sunless tanning compositions that can be used in consumer sunless tanning compositions and/or with commercial-grade sunless tanning booths and/or airbrush machines with improved color and longevity performance.

### SUMMARY

In one aspect of the present disclosure, sprayable sunless tanning compositions are described which may be used by consumers or in commercial settings.

In another aspect, compositions of the present disclosure employ silicone in an aqueous-based sprayable sunless tanning composition. In further aspects, both water dispersible and water soluble silicone-based polymers are used in embodiments of the sunless tanning compositions.

In certain aspects, a water dispersible and a water soluble silicone-based polymer may be used, the combination of which may allow for their use, without the usual blocking effect of silicone cosmetics being observed, and may help to seal the sunless tanning agents on the skin and/or provide a protective layer to one or more sunless tanning agents while still remaining permeable to atmospheric oxygen (O₂). Other beneficial attributes of the compositions of the present disclosure include, but are not limited to better color development and/or longer-lasting color.

Surprising, in contrast to traditional wisdom, certain sunless tanning compositions with both water soluble and water insoluble silicone placed into an aqueous solution can be made in sprayable forms which enhance the delivery and effect of active sunless tanning agents such as dihydroxyacetone (“DHA”), erythrulose, and/or sunless colorants to the skin, for example providing an efficiency equivalent to what previously required a higher amount of the active ingredient to achieve. The improved delivery of sunless tanning agents and the associated formulas may also include benefits such as more uniform deposition, a reduction in the risk of rubbing off of the sunless tanning agents and/or other cosmetic ingredients, and/or beneficial rheological properties when applied to the skin.

In some aspects, sunless tanning compositions of the present disclosure are suitable for commercial spray apparatus, machines, and/or booths. To be suitable for such use, the composition must have certain attributes. For example, such sunless tanning compositions preferably have a particle size appropriate for spray applications, preferably comprise a single phase, do not separate over time, do not contain oils, and/or have significant storage stability over time. In certain aspects, the sunless tanning compositions are suitable for spraying on to the skin and can be delivered in consumer packaging or using a commercial spray apparatus. In other aspects, the sunless tanning composition of the present disclosure is suitable for use in consumer market products including, but not limited to “bag on valve” or “BOV” packaged consumer products.

In additional aspects, the sunless tanning compositions described herein may have a reduced tacky and/or sticky feeling once applied to the skin.

In yet other aspects, the sunless tanning compositions described herein may comprise caramel colorants.

Additional embodiments of the disclosure, as well as features and advantages thereof, will be apparent from the descriptions herein.

### DETAILED DESCRIPTION

For the purposes of promoting an understanding of the principles of the disclosure, reference will now be made to certain embodiments and specific language will be used to describe the same. It will nevertheless be understood that no limitation of the scope of the disclosure is thereby intended, such alterations and further modifications, and such further applications of the principles of the disclosure as described herein being contemplated as would normally occur to one skilled in the art to which the disclosure relates. Additionally, in the detailed description below, numerous alternatives are given for various features. It will be understood that each such disclosed alternative, or combinations of such alternatives, can be combined with the more generalized features discussed in the Summary above, or set forth in the embodiments described below to provide additional disclosed embodiments herein.

As used herein, the term “immediately upon application” or “immediate” generally refers to a time period significantly less than 24 hours, including, for example 15-30 minutes after application. As used herein, the term “long term” and/or “over time” generally refers to a time period greater than about 24 hours, preferably at least about three to about five days, and more preferably at least about five days to about seven days.

Embodiments of the present disclosure may contain one or more sunless tanning agents as active ingredients. Sunless tanning agents typically help the skin develop color over a period of time after application rather than imparting an immediate color. The color preferably lasts for a long term period of time. Examples of suitable sunless tanning agents include, but are not limited to, dihydroxyacetone (“DHA”) and erythrulose, henna and black walnut. Sunless tanning agents may be used in embodiments of the present disclosure in combination with other tanning agents or ingredients.

One or more sunless tanning agents may be present as active ingredients in compositions of the present disclosure. For example, a combination of dihydroxyacetone and erythrulose may be used. In certain embodiments, two, three, four, five, six, or more sunless tanning agents may be used in combination with each other. The ratio of one sunless tanning agents to the other or others may be any suitable ratio. In one embodiment of the present disclosure, dihydroxyacetone is present in the ratio of about 150 to about 1 by weight of erythrulose. In another embodiment dihydroxyacetone is present in the ratio of about 130 to about 1 by weight of erythrulose. In still another embodiment, dihydroxyacetone is present in the ratio of about 75 to about 1 by weight of erythrulose. In yet another example, dihydroxyacetone is present in a ratio of about 1 to about 1 by weight of erythrulose.

In certain embodiments, dihydroxyacetone (DHA) may be present between about 0.1% by weight and about 25% by weight of the composition, between about 5% and 20% by weight, between about 13-19% by weight of the composition, and between about 13-15% by weight of the composition. In some embodiments, when erythrulose is employed, erythrulose may be present between about 0.01% by weight and about 5% by weight of the composition, between about 0.1% and 2.5% by weight, between about 1% by weight and about 2% by weight of the composition, and/or between about 4 to about 5% by weight of the composition.

In certain preferred embodiments, the compositions of the present disclosure preferably comprise one or more silicon-containing polymers. Sunless tanning compositions according to the present disclosure comprise a water-soluble silicone polymer and a water-dispersible silicone polymer placed into an aqueous solution.

Silicone-containing polymers that are water-soluble, and which are suitable for use in embodiments of the present disclosure include, but are not limited to Cetyl Peg/Ppg-10/1 Dimethicone, Peg/PPG-18/18 Dimethicone, Peg/Ppg-20/15 Dimethicone, Peg-12 Dimethicone, Peg-8 Dimethicone.

Silicone-containing polymers that are not soluble in water include, but are not limited to C30-45 Alkyl Cetearyl Dimethicone Crosspolymer, Caprylyl Methicone, Cyclohexasiloxane, Cyclomethicone, Cyclopentasiloxane, Cyclotetrasiloxane, Dimethicone, Dimethicone Crosspolymer, Dimethic one Crosspolymer-3, Dimethicone/Vinyl Dimethicone Crosspolymer, Dimethiconol, Phenyl Trimethicone, Polymethylsilsesquioxane, Stearoxymethicone/Dimethicone Crosspolymer, Stearyl Dimethicone.

In still other embodiments of the present disclosure, a mixture of silicone-containing polymers may be used.

Mixtures of silicone-containing polymers that are water-soluble include, but are not limited to mixtures of Dimethicone, Water (Aqua), Dimethiconol, Laureth-4, Laureth-23, Sodium Salicylate, Ethylparaben, Disodium Edta; and/or mixtures of Sodium Polyacrylate, Dimethicone, Cyclopentasiloxane, Trideceth-6, Peg/Ppg-18/18 Dimethicone, Cyclotestrasiloxane, Water (Aqua).

Mixtures of silicone-containing polymers that are not soluble in water include, but are not limited to mixtures of Cyclomethicone, Dimethicone Crosspolymer-3; mixtures of Cyclopentasiloxane, C30-45 Alkyl Cetearyl Dimethicone Crosspolymer, Cyclomethicone, Cyclotetrasiloxane; mixtures of Cyclopentasiloxane, Cyclohexasiloxane; mixtures of Cyclopentasiloxane, Cyclotetrasiloxane; mixtures of Cyclopentasiloxane, Dimethicone/Vinyl Dimethicone Crosspolymer; mixtures of Cyclopentasiloxane, Dimethiconol, mixtures of Cyclopentasiloxane, Polyethylene, Peg/Ppg-20/15 Dimethicone, Phenyl Methicone; mixtures of Cyclopentasiloxane, Stearoxymethicone/Dimethicone Copolymer, Dimethicone; mixtures of Cyclotetrasiloxane, Cyclopentasiloxane; mixtures of Dimethicone Crosspolymer, Silica; mixtures of Dimethicone, Dimethicone Crosspolymer, Cyclotetrasiloxane; and/or mixtures of Dimethicone/Vinyl Dimethicone Crosspolymer, Silica.

The total concentration of silicone-containing polymers may comprise between about 0.05% (w/w) and about 5% (w/w). In other embodiments, the total concentration of silicone-containing polymers may comprise between about 0.1% (w/w) and about 4% (w/w). In still other embodiments, a single silicone-containing polymer may comprise between about 0.005% (w/w) and about 3% (w/w) of the total composition. In a preferred embodiment, a single silicone-containing polymer may comprise between about 0.1% (w/w) and about 0.5% (w/w) of the total composition.

When sprayable compositions are used that comprise both water dispersible and water soluble polymers in an aqueous solution, it has unexpectedly been observed that a reduced level of dihydroxyacetone and/or erythrulose may be utilized to achieve the same or similar color results as previously required a higher level of sunless tanning agents. For example, a sunless tanning composition with a mixture of silicone-containing polymers included 11% (w/w) DHA. The results were found to meet or exceed the color development in a sunless tanning composition comprising about 13% (w/w) to about 14% (w/w) without silicon-containing polymers.

To be sprayable, the sunless tanning compositions must be suitable for being propelled through a nozzle with a particle size and pressure to be effectively delivered and deposited on the skin at a suitable application distance. In selected embodiments, sprayable formulas herein can be delivered using a compressed gas delivery method such as a BOV apparatus. As representative examples, the BOV apparatus may deliver particles with a volume mean diameter between about 60 to about 90 microns. The BOV apparatus may deliver at least 50% of the particles with a particle size less than between 80 to 90 microns and at least 90% of the particles with a particle size less than between about 110 to 120 microns. The BOV apparatus can be measured at 15 cm from the nozzle, at a pressure between about 3.55 bar and 6.0 bar.

Embodiments of the present disclosure have improved stability over time. The stability of cosmetic formulations is important for commercially significant reasons. For example, commercial consumers may prefer a homogenous formulation that does not separate over time. Phase stability may also be important in “bag on valve” or “BOV”-packaged consumer products. Homogeneity may be measured by storing a formulation in a fluid-tight container for an amount of time and observing if phase separation occurs. For example a formulation preferably may be stored in a stationary state in a fluid-tight container for greater than one month, for greater than three months, for greater than six months, and/or for greater than 12 months.

Sunless tanning compositions according to any of the embodiments may comprise preservatives, one or more antioxidants, one or more pH modifiers, one or more pH buffer systems, one or more fragrances, one or more thickening agents, one or more emulsifying agents, one or more antifungal agents, one or more antimicrobial agents, one or more humectants, one or more emollients, one or more surfactants, one or more sunscreens, and/or one or more solvents. The total quantity of any one or more additives may be any suitable quantity as would be employed by those of ordinary skill in the art.

Various moisturizing agents or humectants that may be included in embodiments of sunless tanning compositions include, but are not limited to, amino acids, chondroitin sulfate, diglycerin, erythritol, fructose, glucose, glycerin, glycerol polymers, glycol, dimethyl isosorbide, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol, hydrogenated castor oil, PEG-40 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

The color as well as other traits of human skin can vary widely. For example, some individuals have a light skin tone while others have a darker skin tone. The skin of some individuals may have more pink, yellow, and/or green undertones present while others have less. Correspondingly, sunless solutions react differently with different skin types. Therefore, it may be desirable to have sunless tanning compositions with different shades of color and/or color intensity. Formulations may be color-balanced with ingredients that contribute differently to the color spectrum and formulations may be designed to match the natural tint of suntanned skinned by altering the ratios of ingredients or colorants. For example, formulations may be designed to produce a light, medium or dark suntanned appearance. The color intensity and/or color spectrum imparted to a particular user can be controlled by selecting certain colorants or color enhancers, in combinations among themselves and/or with other ingredients, to produce a desired net color value or color intensity such as close to that of naturally suntanned skin.

Caramel colorants may be used in certain embodiments of the present disclosure. Caramels are often prepared by heating carbohydrates, in the presence of acids, alkalis, or salts. The chemical products of caramel production are varied and can change based upon the processes used to produce the caramel. Caramel colorants are commonly classified in the industry based upon the processes used to produce the caramel. These classes are summarized below in Table 1.

TABLE 1
Classes of caramel colorants.

| | INS | E | | Restrictions On | |
|---|---|---|---|---|---|
| Class | No. | Number | Description | Preparation | Common uses |
| I | 150a | E150a | Plain caramel, | No ammonium or | Often used in |
| | | | caustic caramel, | sulfite compounds can | whisky or other |
| | | | spirit caramel; | be used during the | high proof |
| | | | | preparation. | alcohols. |
| II | 150b | E150b | Caustic sulfite | Sulfite compounds can | Often used in |
| | | | caramel; | be used but no | cognac, sherry, |
| | | | | ammonium compounds | or vinegars. |
| | | | | can be present | |
| III | 150c | E150c | Ammonia caramel, | ammonium compounds | Often used in |
| | | | baker's caramel, | may be used, but no | beer, sauces, or |
| | | | confectioner's | sulfite compounds may | confections. |
| | | | caramel, beer | be present. | |
| | | | caramel; | | |
| IV | 150d | E150d | Sulfite ammonia | Both ammonium | Often used in |
| | | | caramel, acid-proof | compounds and sulfite | acidic |
| | | | caramel, soft-drink | compounds are present. | environments |
| | | | caramel; | | such as soft |
| | | | | | drinks. |

Two exemplary caramel colorants that may be used in embodiments of the present disclosure include DSL4, a Class IV caramel, and/or SC105, a Class I caramel, both marketed by Sethness Products Company (Skokie, Ill.).

Table 2 summarizes the physical properties of DSL4 and its corresponding physical properties that may be used in various embodiments of the present disclosure. These physical properties include the classification of the caramel, the tinctorial power, the baume of the caramel, the specific gravity of the caramel, the density of the caramel, the pH of the caramel, the typical color intensity of the caramel, and/or the quantity of 4-MEI present in the caramel. Other class IV caramel colorants may be used in certain embodiments based on having one or more similar physical properties, such as a low 4-MEI concentration.

TABLE 2
Summary of physical properties of Class IV caramel colorants
used in certain embodiments of the present disclosure.

| Caramel Colorant | Physical Properties | |
|---|---|---|
| DSL4 (Sethness Products | Class | IV |
| Company of Skokie, IL) | Tinctorial Power, K_{0.56} | 0.370-0.410 |
| | Baume @ 60° F. | 30.7-31.7 |
| | Specific Gravity @ 60° F. | 1.268-1.280 |
| | Pounds per gallon @ 60° F. | 10.56-10.66 |
| | pH | 2.5-3.0 |
| | Typical Color Intensity | 0.230-0.258 |
| | 4-MEI | <30 ppm |

Class IV caramel colorants are produced from carbohydrates that are heated in the presence of both sulfite and ammonium compounds. In certain embodiments, the Class IV caramel colorants may have an isoelectric point between pH 0.5 and 2, and may carry a negative ionic charge above pH 2.

When caramel colorants are used, such caramel colorants will typically have a concentration between greater than about 0% (w/w) and less than or equal to about 15% (w/w). In additional embodiments, when caramel colorants are used, these colorants have a concentration between about 0.05% (w/w) and 13% (w/w). In still other preferred embodiments, when caramel is used as a colorant, the total caramel present is between about 4% (w/w) and about 5% (w/w).

Other colorants may be used in embodiments of the present disclosure. Certain colorants are extracts such as from beet, rosemary, annatto, saffron, turmeric, turmeric root, purple sweet potato, cochineal, carrots, wheat, corn, pepper, spirulina, chlorophyll, red cabbage, and grape skin. Other colorants are dyes such as, but not limited to, Red 4, Red 33, Red 40, Carmine, Blue 1, Yellow 5, and/or carbon black.

Embodiments of the disclosed sunless tanning compositions may be applied to the skin of a human by spraying. In some embodiments, application may be performed by commercial spray apparatus, machines, and/or booths, or alternatively aerosol and/or pump spray mechanisms may be utilized. Certain embodiments of the disclosed sunless tanning compositions may be applied to the skin using a “bag on valve” or “BOV”-packaged consumer products.

Compositions of the present disclosure preferably exist as solutions, for example, where the composition exists as a single continuous phase. Any suitable vehicle may be used in embodiments of the present disclosure, such suitable vehicles including cosmetically acceptable vehicles. Cosmetically acceptable vehicles may comprise, for example, but not limited to, water.

In order to promote a further understanding of the present disclosure and its various embodiments, the following specific examples are provided. It will be understood that these examples are illustrative and not limiting of the disclosure.

### EXAMPLE 1

### Preparation of a Sunless Tanning Composition

### Materials and Methods:

A composition comprising the ingredients listed in Table 3 was prepared to comprise: water, disodium EDTA, caffeine, melanin, dihydroxyacetone, propylene glycol, erythrulose, sodium benzoate, potassium sorbate, Saccharomyces Ferment, and Witch Hazel Water was prepared according to procedures known to those of skill in the art. Citric acid was then added to this composition. PEG-12 dimethicone, fragrance, PEG-40 hydrogenated castor oil, and polysorbate 20 was then added to the resulting composition. Caramel color comprising SC104 from Sethness Products Company (Skokie, Ill.), and/or DSL4 from Sethness Products Company (Skokie, Ill.) was then added. Finally, a composition comprising dimethicone, water, dimethiconol, laureth-4, laureth-23, phenoxyethanol, caprylyl glycol, potassium sorbate, and hexylene glycol was added.

TABLE 3
Sunless Tanning Composition of EXAMPLE 1.

| | Percentage of Final |
|---|---|
| Ingredient | Composition (w/w) |
| Dihydroxyacetone | 7.0% |
| Erythrulose | 0.05% |
| Disodium EDTA | 0.1% |
| Caffeine | 0.1% |
| Melanin | 0.05% |
| Propylene glycol | 6.0% |
| Water, Sodium Benzoate, | 0.2% |
| Potassium Sorbate | |
| Saccharomyces Ferment, | 0.2% |
| Potassium Sorbate, Sodium | |
| Benzoate | |
| Hammamelis Virginiana | 5.0% |
| (Witch Hazel) Water, Alcohol | |
| Citric Acid | 0.78% |
| PEG-12 Dimethicone | 0.8% |
| Fragrance | 0.2% |
| PEG-40 Hydrogenated Castor | 0.25% |
| Oil | |
| Polysorbate 20 | 0.9% |
| SC105 Caramel | 3.52% |
| DSL4 Caramel | 1.24% |
| Dimethicone, water, | 0.75% |
| Dimethiconol, Laureth-4, | |
| Laureth-23, Phenoxyethanol, | |
| Caprylyl Glycol, Potassium | |
| Sorbate, Hexylene Glycol | |
| Purified Water | Remainder |

### Results:

The composition prepared according to the procedure described above was observed to be a single phase.

When applied to the skin of a human, the composition prepared according to the procedure describe above subjectively felt less tacky on the skin than other sunless tanning compositions.

The uses of the terms “a” and “an” and “the” and similar references in the context of describing the disclosure are to be construed to cover both the singular and the plural unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

While the disclosure has been illustrated and described in detail in the foregoing description, the same is to be considered as illustrative and not restrictive in character, it being understood that only the preferred embodiment has been shown and described and that all changes and modifications that come within the spirit of the disclosure are desired to be protected. In addition, all references cited herein are indicative of the level of skill in the art and are hereby incorporated by reference in their entirety.

What is claimed is:

1. A method for improving the color imparted to human skin from a sunless tanning composition of matter, comprising the acts of:
providing one or more water dispersible polymers, and one or more water soluble silicone polymers in said sunless tanning composition and optionally one or more humectants in said sunless tanning composition;
providing dihydroxyacetone or erythrulose in said sunless tanning composition;
providing a nozzle through which said sunless tanning composition can be sprayed; and
spraying the sunless tanning composition of matter on human skin.

2. The method of claim 1, wherein said spraying is performed by a commercial spraying apparatus at a salon.

3. The method of claim 1, wherein said spraying is performed using a bag on valve system.

4. The method of claim 1, wherein the act of spraying is performed so that at least 50% of the particles have a particle size less than between 80 to 90 microns and at least 90% of the particles with a particle size less than between about 110 to 120 microns when the particle size is measured at 15 cm from the nozzle, at a pressure between about 3.55 bar and 6.0 bar.

5. A sprayable sunless tanning composition of matter comprising:
a sunless tanning agent
a silicone polymer wherein said silicone polymer comprises one or more water dispersible polymers, and one or more water soluble silicone polymers; and
wherein said sprayable sunless tanning composition can be applied by spraying.

6. The sprayable sunless tanning composition of matter of claim 5, wherein said sunless tanning agent comprises dihydroxyacetone or erythrulose.

7. The sunless tanning composition of claim 5, wherein said water soluble silicone polymers comprises one or more Dimethicone; or mixtures of Sodium Polyacrylate, Dimethicone, Cyclopentasiloxane, Trideceth-6, Peg/Ppg-18/18 Dimethicone, Cyclotestrasiloxane, and Water (Aqua).

8. The sunless tanning composition of claim 5, wherein said water dispersible polymers comprises one or more of Cyclomethicone, Dimethicone Crosspolymer-3; mixtures of Cyclopentasiloxane, C30-45 Alkyl Cetearyl Dimethicone Crosspolymer, Cyclomethicone, Cyclotetrasiloxane; mixtures of Cyclopentasiloxane, Cyclohexasiloxane; mixtures of Cyclopentasiloxane, Cyclotetrasiloxane; mixtures of Cyclopentasiloxane, Dimethicone/Vinyl Dimethicone Crosspolymer; mixtures of Cyclopentasiloxane, Dimethiconol, mixtures of Cyclopentasiloxane, Polyethylene, Peg/Ppg-20/15 Dimethicone, Phenyl Methicone; mixtures of Cyclopentasiloxane, Stearoxymethicone/Dimethicone Copolymer, Dimethicone; mixtures of Cyclotetrasiloxane, Cyclopentasiloxane; mixtures of Dimethicone Crosspolymer, Silica; mixtures of Dimethicone, Dimethicone Crosspolymer, Cyclotetrasiloxane; or mixtures of Dimethicone/Vinyl Dimethicone Crosspolymer, Silica.

9. The composition of matter of claim 5, wherein said silicone polymer is present in about 0.05% (w/w) to about 4% (w/w) of the total composition, in about 0.005% (w/w) to about 3% (w/w) or about 0.1% (w/w) to about 0.5% (w/w).

10. The composition of matter of claim 6, wherein said dihydroxyacetone is present in about 0.1% (w/w) to about 25% (w/w), about 5% (w/w) to about 20% (w/w), about 13% (w/w) to about 19% (w/w), or about 13% (w/w) to about 15% (w/w).

11. The composition of matter of claim 5, further comprising one or more humectants.

12. The composition of matter of claim 11, wherein said one or more humectants are amino acids, chondroitin sulfate, diglycerin, erythritol, fructose, glucose, glycerin, glycerol polymers, glycol, dimethyl isosorbide, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol, hydrogenated castor oil, PEG-40 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, or ylang ylang (cananga odorata) oil.

13. The sunless tanning composition of matter of claim 5, further comprising caramel.

14. The sunless tanning composition of matter of claim 13, wherein said caramel is a DSL4, a Class IV caramel.

15. The sunless tanning composition of matter of claim 14, wherein said caramel is a SC105, a Class I caramel.

16. The composition of matter of claim 13, wherein said caramel comprises<30 ppm 4-MEI.

17. The composition of matter of claim 10, wherein said silicone polymer is present in about 0.05% (w/w) to about 4% (w/w) of the total composition, in about 0.005% (w/w) to about 3% (w/w) or about 0.1% (w/w) to about 0.5% (w/w).
